SEQUENCE LISTING (32 sequences)

SEQ ID NO: 1 - protein, 8 aa, Homo sapiens
MDRGSDGK

SEQ ID NO: 2 - protein, 8 aa, Homo sapiens
MESRADRK

SEQ ID NO: 3 - protein, 8 aa, Homo sapiens
MDRTPDGR

SEQ ID NO: 4 - protein, 8 aa, Homo sapiens
MEREFESN

SEQ ID NO: 5 - protein, 8 aa, Homo sapiens
CTTRTDTC

SEQ ID NO: 6 - protein, 8 aa, Homo sapiens
CERTTEGC

SEQ ID NO: 7 - protein, 8 aa, Homo sapiens
CTTSTDSC

SEQ ID NO: 8 - protein, 8 aa, Homo sapiens
CSLDTSTC

SEQ ID NO: 9 - protein, 8 aa, Homo sapiens
CSTDISTC

SEQ ID NO: 10 - protein, 8 aa, Homo sapiens
CSGDMDSC

SEQ ID NO: 11 - protein, 8 aa, Homo sapiens
CPLSNATC

SEQ ID NO: 12 - protein, 8 aa, Homo sapiens
  MISC_FEATURE (7)..(7): Xaa = Gln or His
CPSAAAXC

SEQ ID NO: 13 - protein, 7 aa, Homo sapiens
TTRTDTC

SEQ ID NO: 14 - protein, 9 aa, Homo sapiens
YMESRADRK

SEQ ID NO: 15 - protein, 6 aa, Homo sapiens
TTDTRT

SEQ ID NO: 16 - protein, 6 aa, Homo sapiens
TTRTDT

SEQ ID NO: 17 - protein, 9 aa, Homo sapiens
KMDASAAVS

SEQ ID NO: 18 - protein, 10 aa, Homo sapiens
KRFYVVMWKK

SEQ ID NO: 19 - protein, 6 aa, Homo sapiens
GRGDSP

SEQ ID NO: 20 - protein, 18 aa, Homo sapiens
GAPLFMDRGSDGKLQEVG

SEQ ID NO: 21 - protein, 18 aa, Homo sapiens
GAPLYMESRADRKLAEVG

SEQ ID NO: 22 - protein, 18 aa, Homo sapiens
GAPLYMDRTPDGRPQEVG

SEQ ID NO: 23 - protein, 18 aa, Homo sapiens
GAPLFMEREFESNPREVG

SEQ ID NO: 24 - protein, 12 aa, Homo sapiens
GAPMQSTIREEG

SEQ ID NO: 25 - protein, 18 aa, Homo sapiens
GYYCNCTTRTDTCMSSNG

SEQ ID NO: 26 - protein, 18 aa, Homo sapiens
GSACQCERTTEGCLNPRR

SEQ ID NO: 27 - protein, 18 aa, Homo sapiens
GEYCNCTTSTDSCVSEDG

SEQ ID NO: 28 - protein, 18 aa, Homo sapiens
GSACDCSLDTSTCEASNG

SEQ ID NO: 29 - protein, 18 aa, Homo sapiens
GDNCNCSTDISTCRGRDG

SEQ ID NO: 30 - protein, 18 aa, Homo sapiens
GRACECSGDMDSCISPEG

SEQ ID NO: 31 - protein, 18 aa, Homo sapiens
GPSCDCPLSNATCIDSNG

SEQ ID NO: 32 - protein, 18 aa, Homo sapiens
  MISC_FEATURE (12)..(12): Xaa = Gln or His
GDRCQCPSAAAXCVNSKG

(19) United States
(12) Patent Application Publication
(10) Pub. No.: US 2009/0191645 A1
(43) Pub. Date: Jul. 30, 2009

(54) COMPOUNDS AND METHODS FOR MODULATING INTEGRIN ACTIVITY

(75) Inventor: WILLIAM A. FRAZIER, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: Washington University in St. Louis, St. Louis, MO (US)

(21) Appl. No.: 12/353,167

(22) Filed: Jan. 13, 2009

Related U.S. Application Data
(60) Provisional application No. 61/020,831, filed on Jan. 14, 2008.

(51) Int. Cl. G01N 33/566; C07K 7/06; C07K 16/00

(52) U.S. Cl. 436/501; 530/329; 530/328; 530/387.1

(57) ABSTRACT

The present invention provides methods and compositions for modulating integrin activity. In particular, the present invention encompasses methods and compositions for altering the interaction between the α and β chain extracellular clasp regions.

### CROSS REFERENCE TO RELATED APPLICATIONS

This application claims the priority of provisional application No. 61/020,831, filed Jan. 14, 2008, which is hereby incorporated by reference in its entirety.

### GOVERNMENTAL RIGHTS

The present invention was made, at least in part, with support by the National Institutes of Health, National Heart, Lung, and Blood Institute, grant number HL 054392. Accordingly, the United States Government may have certain rights in the invention.

### FIELD OF THE INVENTION

The invention encompasses methods and compositions for modulating the activity of an integrin.

### BACKGROUND OF THE INVENTION

Control of integrin activity is of crucial importance in regulating many fundamental biological processes. These include platelet aggregation in hemostasis, leukocyte adherence and trafficking in the immune system, and cell migration, differentiation and apoptosis during development (1, 2). In recent years tremendous strides have been made in understanding the structural basis for the regulation of integrin ligand binding and the transmission of this event to intracellular signaling cascades. Crystal structures of integrin domains (3) and the structure of the entire extracellular domain of αvβ3 (4, 5) have provided new hypotheses for integrin regulation. However, the manner in which the ligand binding activity and signaling of integrins is related to their structural state is still incompletely understood.

Two general mechanisms regulate the functional state of integrins. Conformational changes of the αβ dimer are clearly involved in transitions from low to high affinity states (6), usually judged by the binding of soluble ligands or the exposure of binding sites for mAbs that recognize an activated conformation stabilized by ligands or a “ligand-induced binding site” (anti-LIBS mAbs) (7-9). Integrin dimers competent for ligand binding may also be clustered resulting in a high “avidity” state that increases binding to multivalent, usually immobilized ligands (10, 11). Clustering may be driven by the valency of the ligands themselves if mobility of the integrin within the membrane is allowed (12). Both of these heightened functional states of the integrin may be modulated by proteins associated with integrins in the plane of the membrane. These include CD47 (integrin-associated protein) (13), the urokinase plasminogen activator receptor (uPAR) (14), CD98 (15) and tetraspannins (4TM) (16).

Much attention has focused on the integrin heterodimer itself in a search for clues to the features regulating activation. Early studies of αIIbβ3 activation suggested that juxtamembrane cytoplasmic ion pairs opposite each other in the α and β subunit tails could lock the transmembrane (TM) regions together thus restraining conformational changes necessary for activation (17) (18). The notion that this cytoplasmic domain “clasp” of the α and β subunits is important in regulating “inside-out” signaling was strengthened by the identification of additional mutants in the juxtamembrane regions of αIIb and β3 that result in constitutive activation (19). Further, the addition of non-native, coiled coil dimerizing peptides to the cytoplasmic tails of α and β subunits constrained activation (20, 21). Recently, mutations in TM domains of α and β subunits have been identified that constitutively activate αIIbβ3 (19). In addition, truncation of the α and β TM segments yields a soluble integrin in a high affinity state (22). This data suggests a model in which a specific α-β TM helix interface contributes to stabilizing the off state, perhaps acting in concert with the juxtamembrane clasp in the cytoplasmic tails.

The publication of the crystal structure of free and RGD-bound extracellular domains of αvβ3 (4, 5), gave rise to an entirely new and still controversial model for activation. The bent or genuflected integrin seen in the crystal structure suggested that massive conformational changes of the entire αvβ3 extracellular domain must accompany integrin activation, if indeed the fully active integrin were to assume the extended, upright conformation expected from earlier EM studies (23-26). The source of controversy here is the uncertainty in what a fully activated integrin should look like. Studies with soluble, truncated β3 integrin constructs show that the bent conformation can indeed bind soluble ligands such as RGD peptides (5) and even fragments of fibronectin (27). Studies by Springer's group support the idea that the bent structure seen in the crystals of αvβ3 is likely the physiologically relevant “off” or low affinity state and an extended, erect integrin represents the fully active state (6, 11, 12, 28). Other experiments have demonstrated that the bent structure as revealed in the model can exist on the cell surface and is in an inactive state. Hence, there exists a need in the art to determine the structural basis for integrin regulation.

### SUMMARY OF THE INVENTION

One aspect of the invention encompasses a compound that modulates the activity of an integrin by altering the interaction between the α and β chain extracellular clasp regions.

Another aspect of the invention encompasses a method of modulating the activity of an integrin. The method comprises altering the interaction of the α and β chain extracellular clasp region of the integrin.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

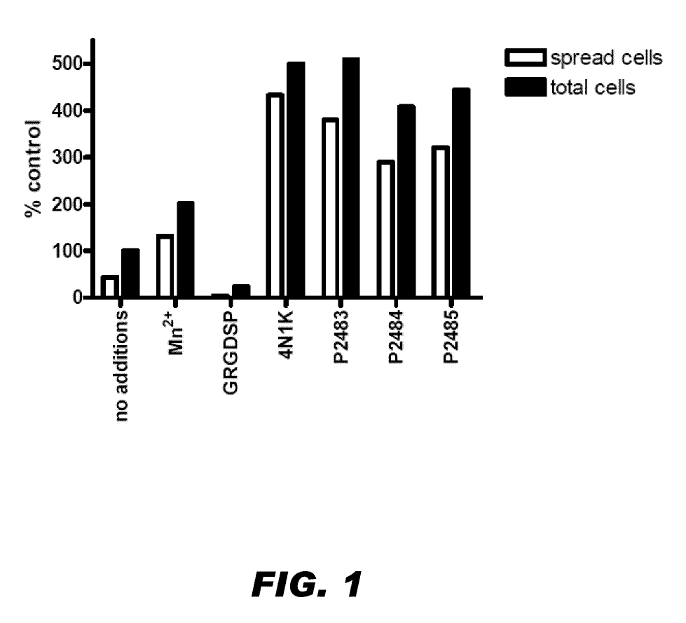

FIG. 1 depicts a graph showing that clasp peptides stimulate C32 cell adhesion and spreading. C32 melanoma cells were treated with the following and then allowed to attach and spread on vitronectin coated tissue culture plates: no treatment, 2 mM MnSO₄, 500 μM GRGDSP (SEQ ID NO: 19), 50 μM 4N1K, 100 μM P2483 (β3 peptide), 100 μM P2484 (αIIb peptide). The open bar represents the total number of adherent cells scored in the assay; the black bar indicates the total number of spread cells. The number of cells adhering with no additions was set at 100%. The experiment was repeated twice with similar results.

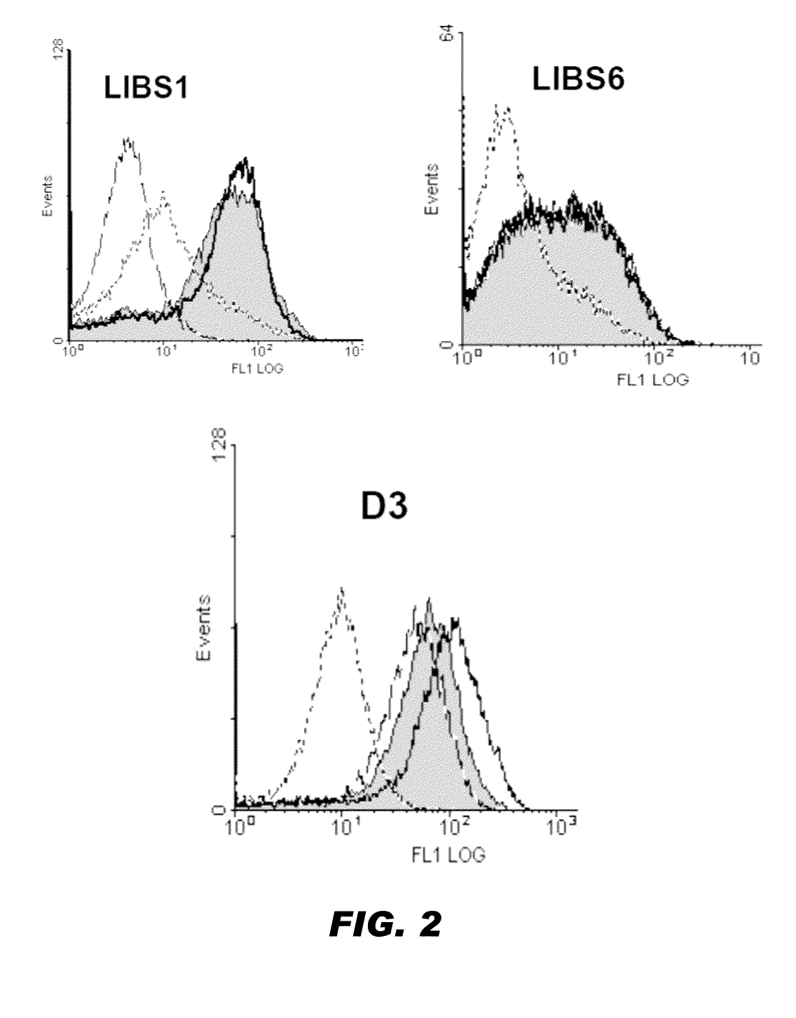

FIG. 2 depicts graphs showing the effect of clasp ceptides on the binding of LIBS antibodies to αvβ3 on K562 cells. Human K562 erythroleukemia cells transfected with WT human αv and β3 cDNAs were incubated on ice with the indicated LIBS mAb (dotted line), plus 1 mM GRGDSP (SEQ ID NO: 19) peptide (dashed line), and peptide 2483 (β3 clasp, shaded histogram) or 2484 (αIIb clasp, solid line). Bound LIBS mAbs were detected with FITC-anti-mouse IgG and quantified on a Coulter EPICS flow cytometer. Each experiment was repeated 3 to 8 times, and results of a representative one are shown.

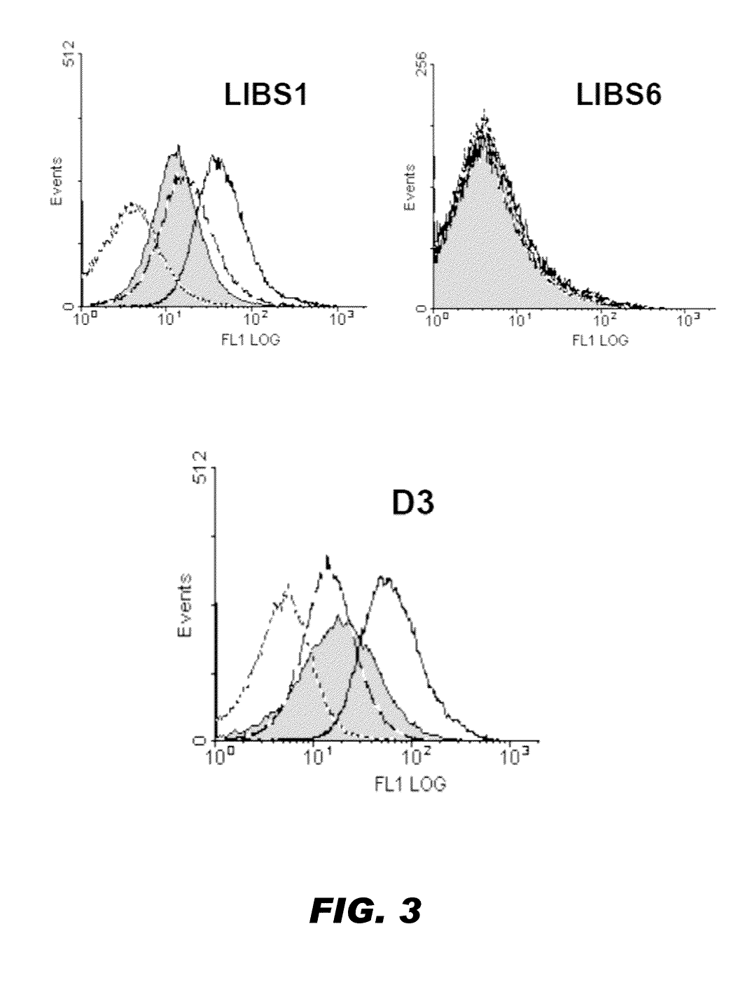

FIG. 3 depicts graphs showing the effect of clasp peptides on the binding of LIBS antibodies to human platelets. Human platelets were collected, washed, incubated on ice overnight, and tested for the effect of clasp peptides on LIBS binding to αIIbβ3 integrin by flow cytometry as in FIG. 2. The same results were obtained with fresh platelets inhibited with PGE1 and apyrase. The experiment was repeated 6 times and a representative one is shown.

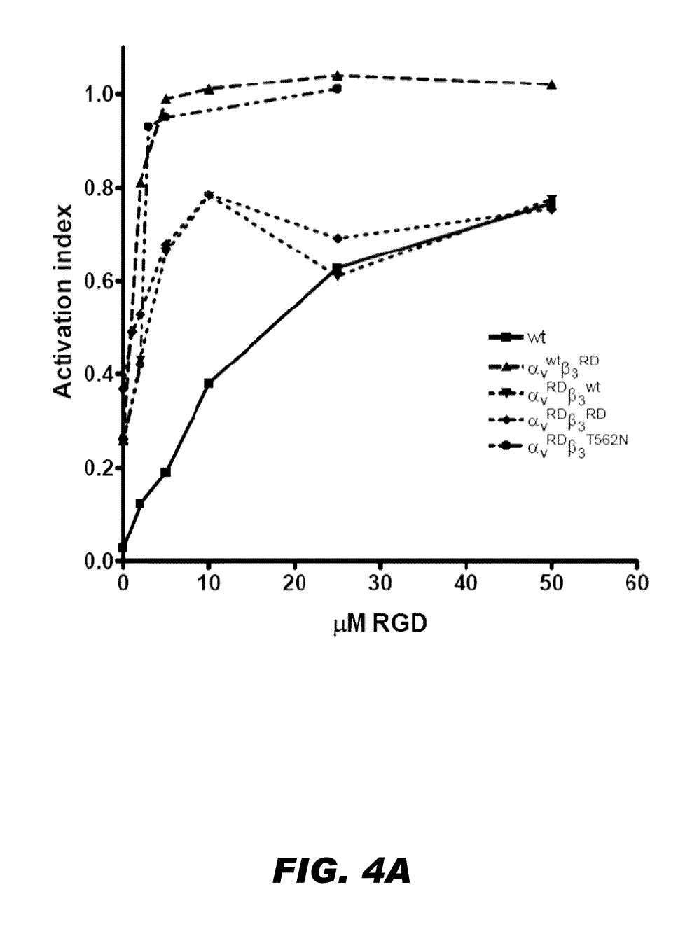

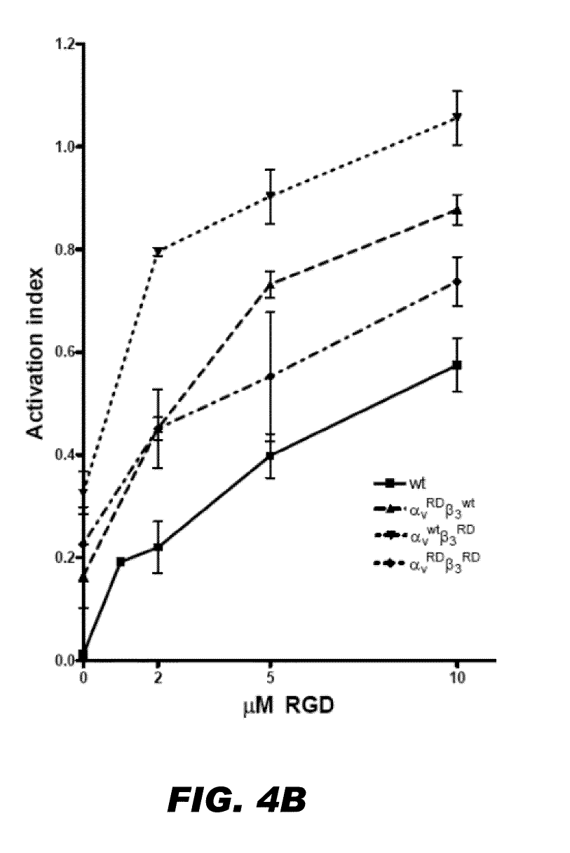

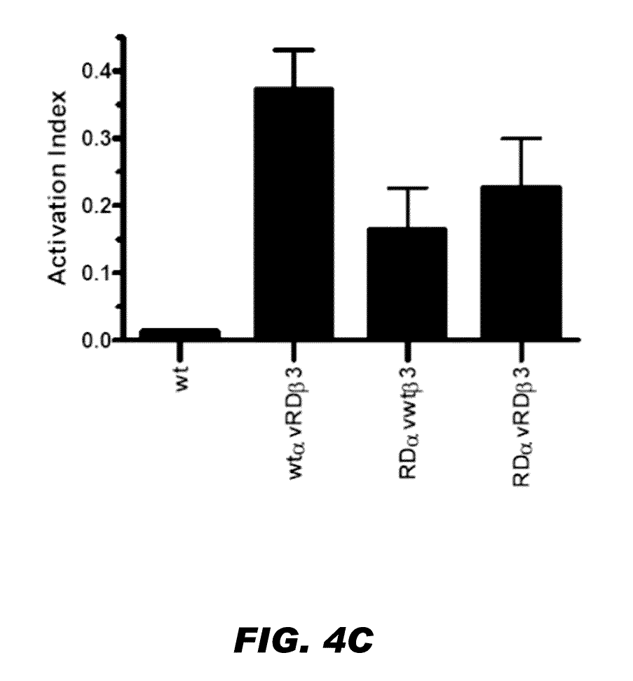

FIG. 4 depicts graphs showing the activation status of αvβ3 clasp mutants. A. The activation state of each mutant integrin was determined by the binding of LIBS D3 as a function of GRGDSP concentration with each value normalized to the maximum D3 binding obtained in 1 mM MnCl₂ and 100 μM GRGDSP (SEQ ID NO: 19). Panel A shows a single representative experiment for each integrin. Panel B presents data pooled from all experiments in the concentration range of 0 to 10 μM RGD peptide to emphasize the activation of the mutants at very low peptide concentrations. Panel C presents pooled data showing the increase in “spontaneous” activation (i.e. binding of D3 with no added RGD peptide) of each mutant and WT integrin. (p values: WT vs αvWTβ3R/D=0.0003; WT vs αvR/Dβ3WT=0.015; WT vs. αvR/Dβ3R/D=0.008; αvWTβ3R/D vs αvR/Dβ3WT=0.036; αvWTβ3R/D vs αvR/Dβ3R/D=0.11).

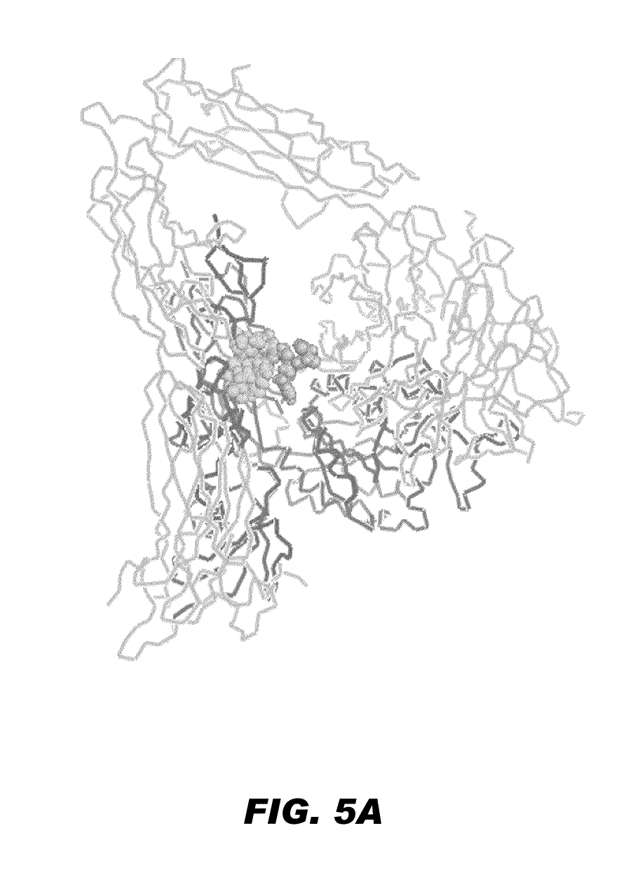

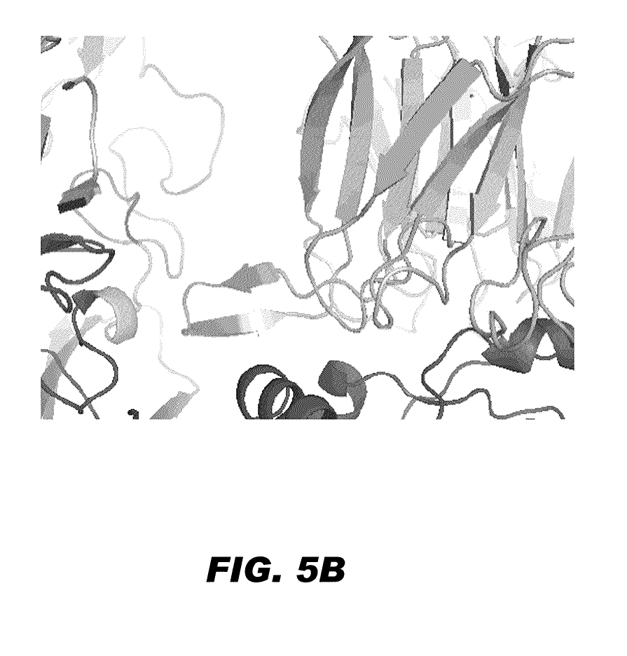

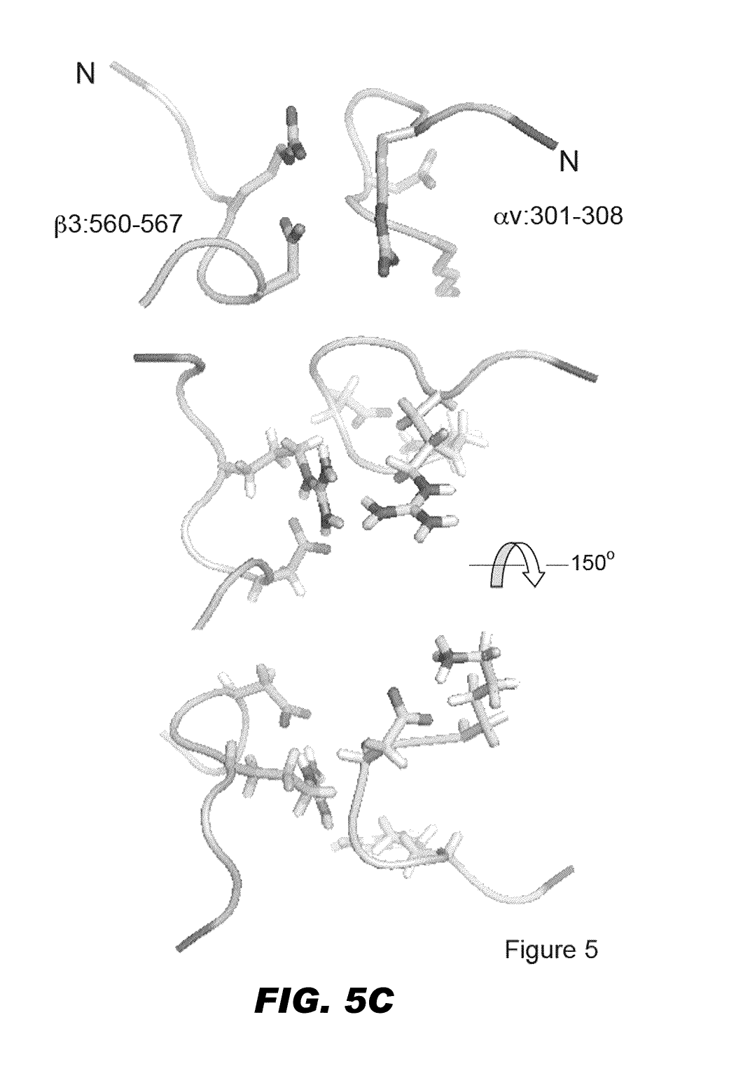

FIG. 5 depicts illustrations showing that the equilibration of the αvβ3 structure reveals a complex clasp interface. A. Backbone representation of αvβ3 integrin rendered with the crystal coordinates (4). The αv subunit is in gold and the β3 in blue. The clasp residues, αv: 301-308 and β3: 561-566 are shown in spacefill as they are located in the crystal structure. B. Zoomed in view of the clasp region of A showing the αv clasp loop (magenta) projecting from a turn between two antiparallel β strands of the αv propeller toward the β3 clasp (cyan). C. TOP: the WT clasp structure (oriented as in A) according to the crystal coordinates which do not specify the positions of hydrogen atoms. MIDDLE: the WT median clasp structure (oriented as above) after addition of hydrogens and equilibration. Note the ring closed by the intrachain pairing of β3-R563 and D565 (cyan). BOTTOM: The clasp as seen in the median equilibrated structure shown in the middle, rotated toward the viewer about 150 degrees to obtain a view of the ring closed by pairing of αv-D306 with K308 (gold).

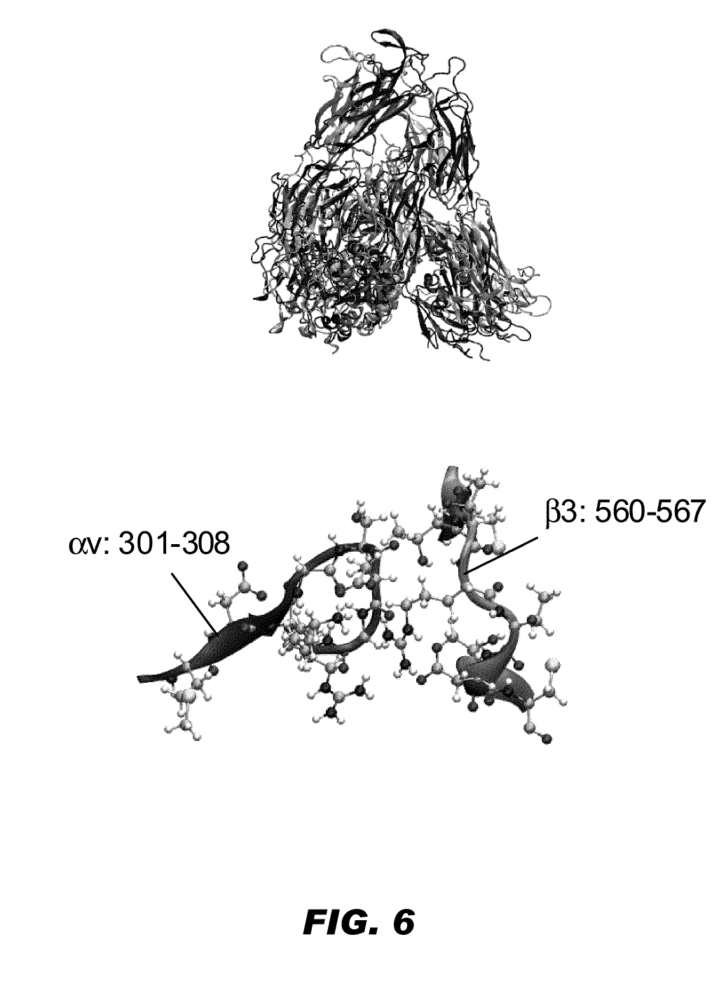

FIG. 6 depicts illustrations of the crystal structure of αvβ3. (a) Crystal structure of αvβ3 and its structure at energy minima state (silver: crystal structure, blue: structure at its energy minima state. (b) Clasp region at its energy minima state (red: a chain, orange: β chain), residues are represented in CPK style. The images were made with VMD software support.

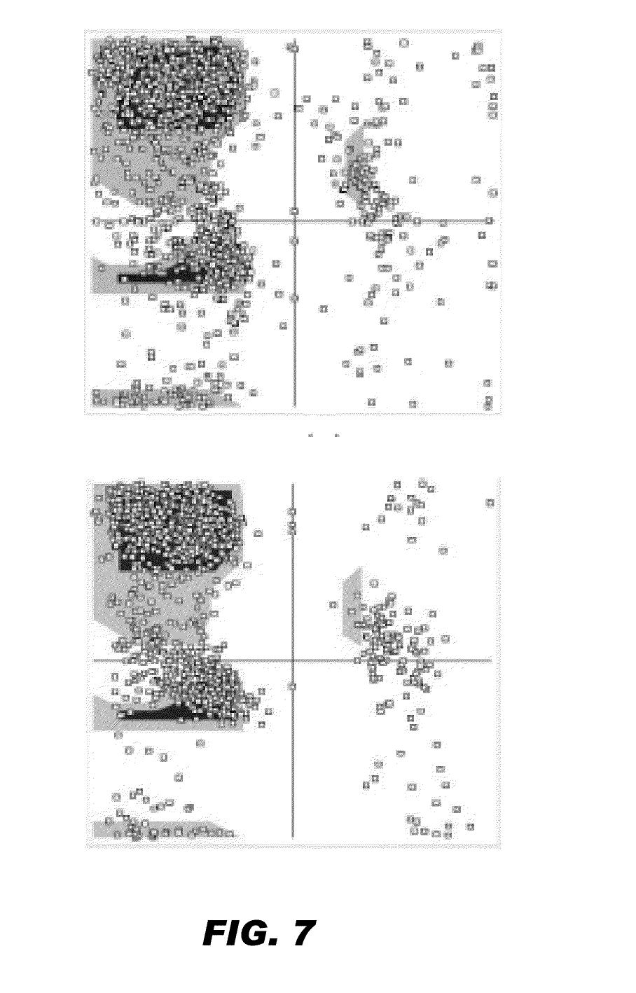

FIG. 7 depicts Ramachandran plots of the crystal structure (a) and the structure obtained after energy minimization (b). Green and blue colored areas are the allowed and favored regions of Ramachandran space respectively. Each gold dot represents a single residue.

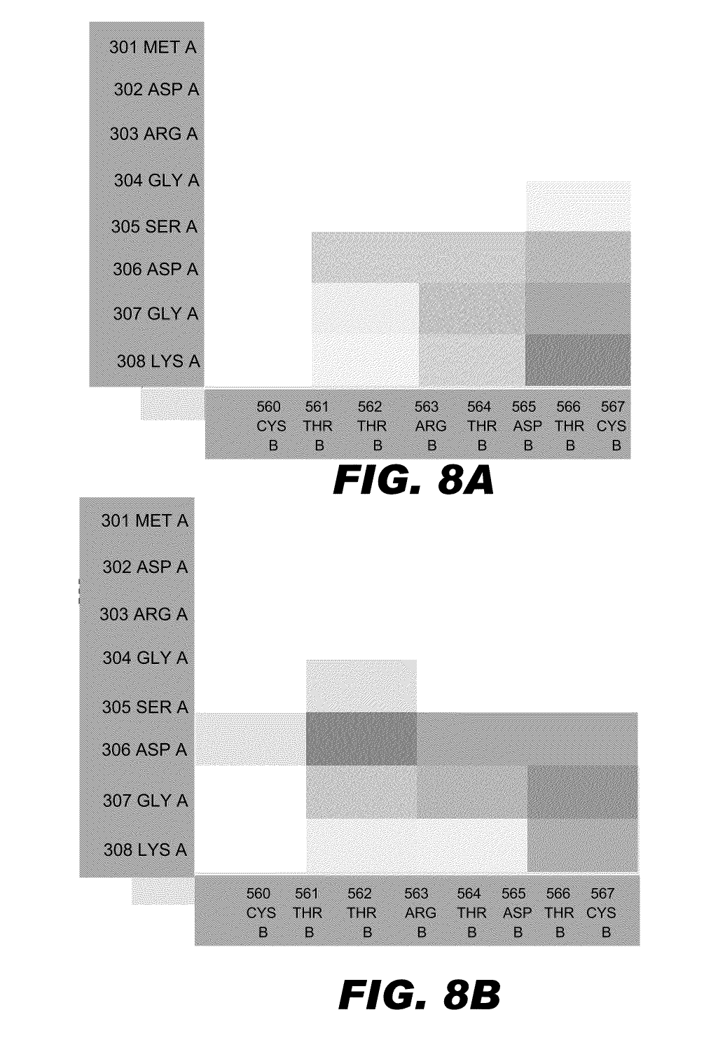

FIG. 8 depicts a contact map for the residues in the clasp region of the αv chain and the residues in the clasp region of the β3 chain. (a) Crystal structure; (b) structure after energy minimization. The X axis is the residue number in the clasp region of the a chain; y axis is the residue number in the clasp region of the β chain. The color-coded matrix shows the contact distances between alpha-Carbons of the paired residues. Darker colors represent residues that are closer to each other and lighter colors represent residue pairs that are more distant from each other. A graph square is colored black at 0.0 Angstrom distance, to a linear gray scale between 0.0 and 20.0 Angstroms. When the distance is equal to or greater than 20.0 Angstroms, the square is white. The images were made with VMD software support.

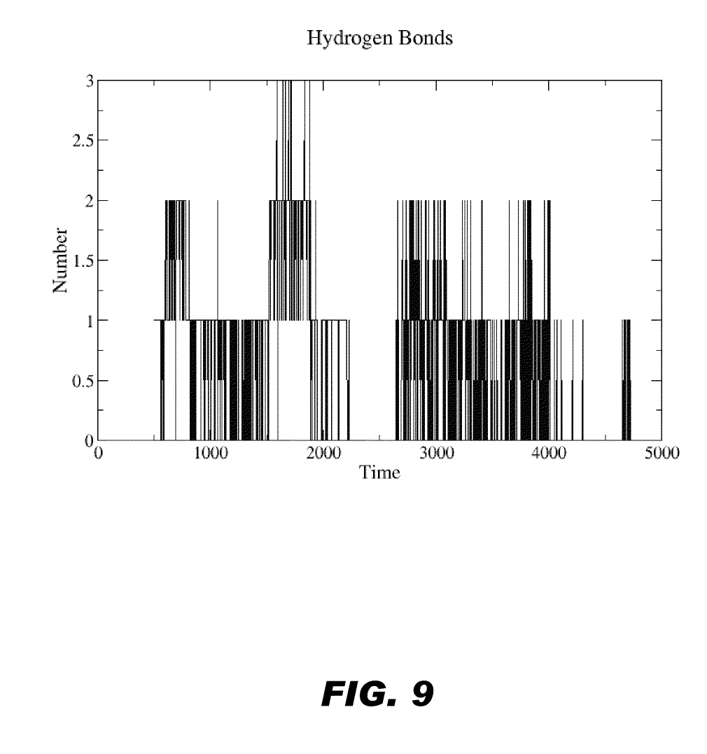

FIG. 9 depicts a graph showing the hydrogen bonds formed between the residues in the clasp region of a chain and the residues in the clasp region of β chain during molecular dynamics simulation.

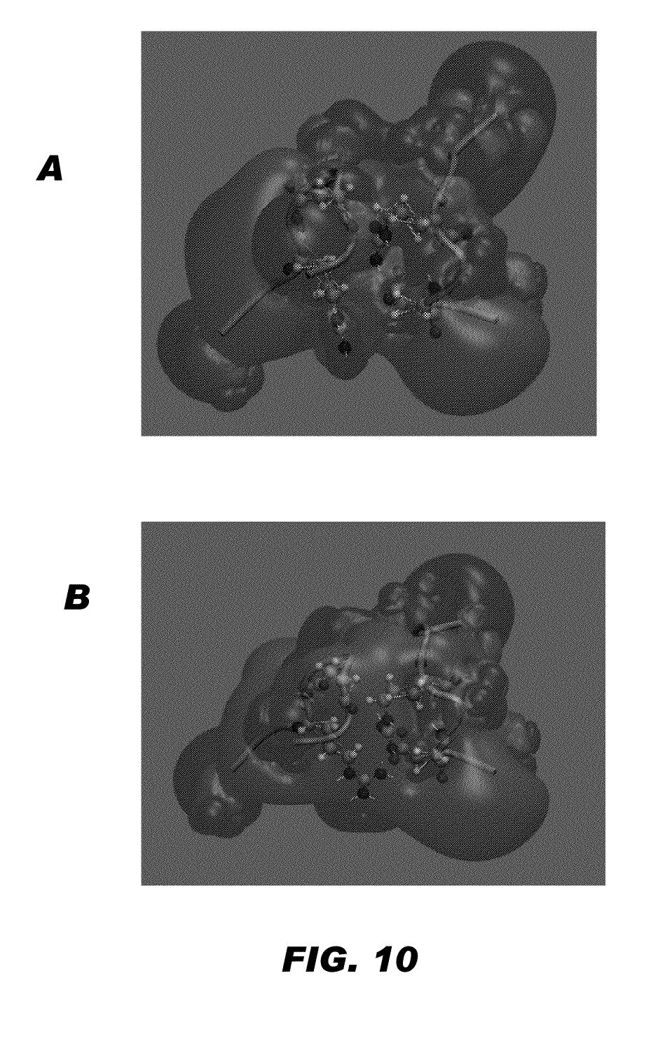

FIG. 10 depicts images showing the electrostatic potential distribution on the molecular surface of the clasp region (blue: positive electrostatic potential; red: negative electrostatic potential; orange ribbon: a chain in the clasp region; cyan: β chain in the clasp region; CPK representations are ARG and ASP residues in the clasp region. (a) Crystal structure; (b) structure after energy minimization.

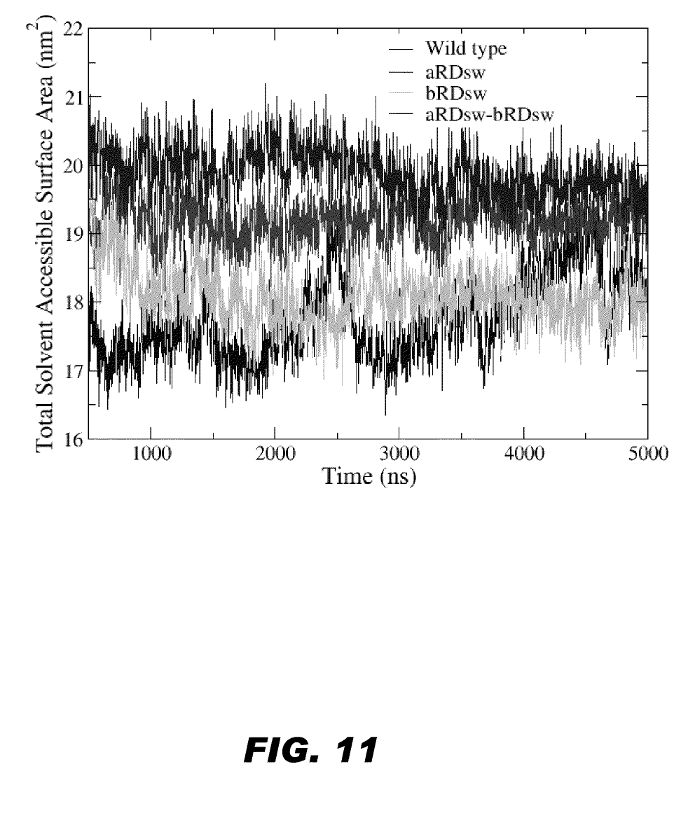

FIG. 11 depicts a graph showing the effect of mutations on the solvent accessible surface area of the clasp region

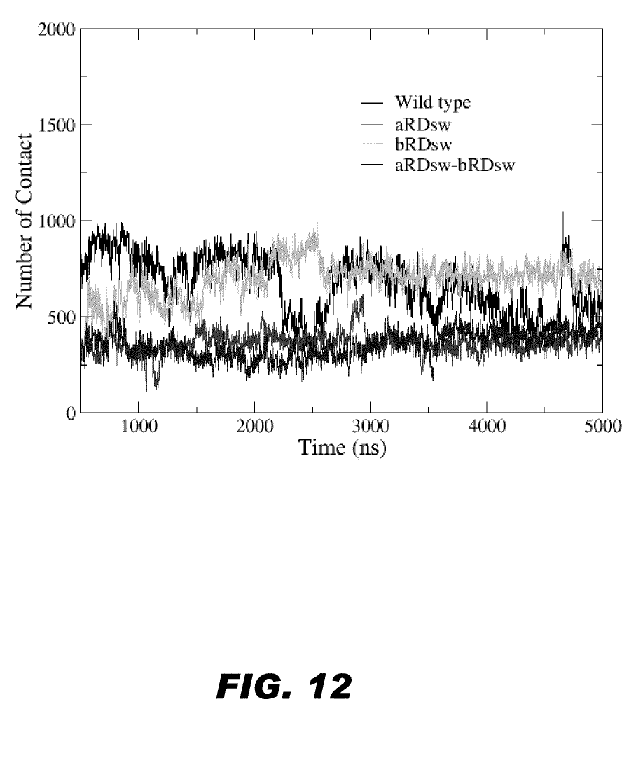

FIG. 12 depicts a graph showing the effect of mutations on the number of contacts at the clasp region.

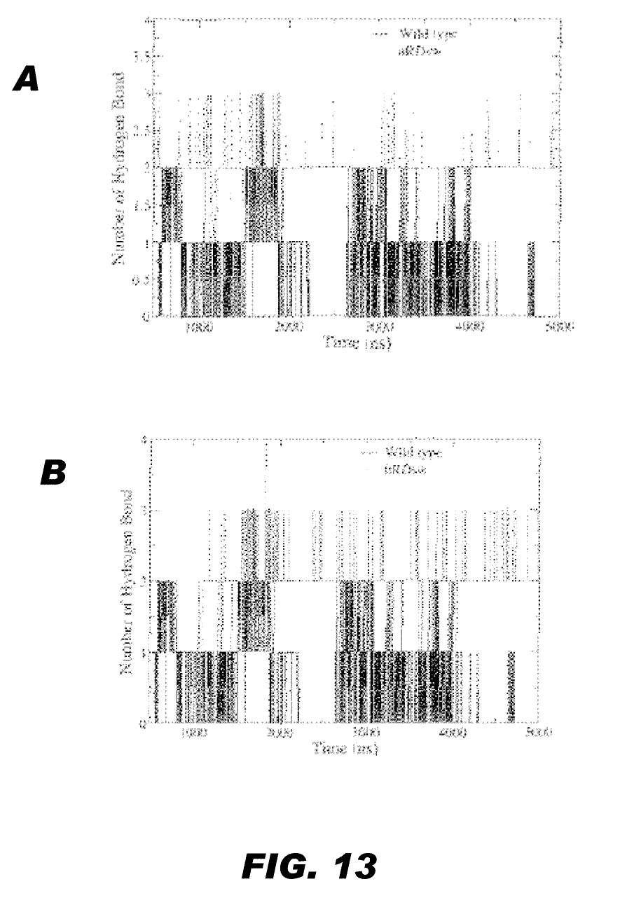

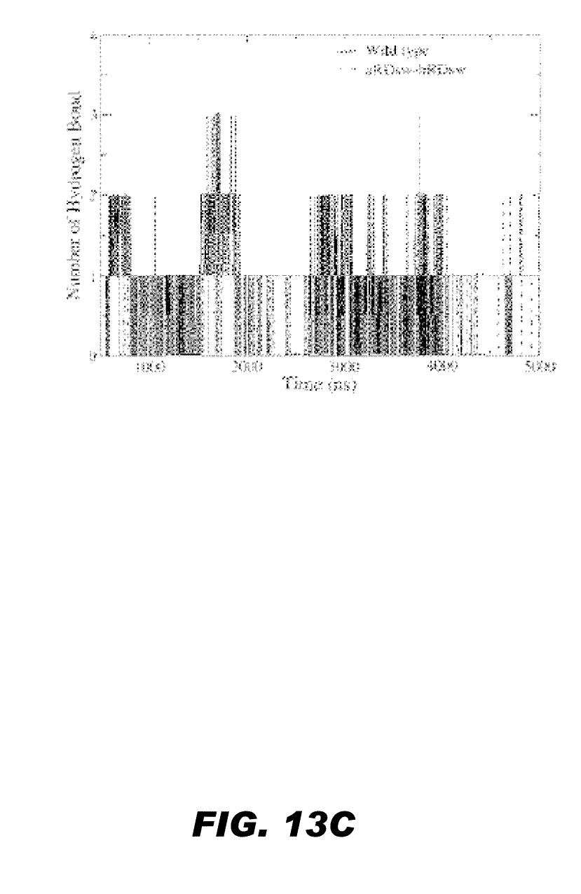

FIG. 13 depicts graphs showing the effect of mutations on hydrogen bond formation at the clasp region. (a) αv-R/D swap, (b) β-R/D swap, (c) double swap (αv-R/D swap and β3-R/D swap).

### DETAILED DESCRIPTION OF INVENTION

The present invention provides methods and compositions for modulating the activity of an integrin. Specifically, the present invention provides methods and compositions for modulating the interaction between the α and β chain extracellular clasp regions of the integrin.

Suitable α chains may include, but are not limited to, αv, αIIb, α5, and α8. Suitable β chains may include, but are not limited to, β1, β2, β3, β4, β5, β6, β7, and β8. Consequently, suitable integrins may include, but are not limited to, αvβ3, αIIbβ3, αLβ2, αMβ2, αXβ2, or integrins listed in Table A.

TABLE A

| α chain | combinations |
|---|---|
| αv | αvβ1, αvβ3, αvβ5, αvβ6 |
| αIIb | αIIbβ3 |
| α5 | α5β1, α5β5, α5β6 |
| α8 | α8β1 |

The extracellular clasp region of an integrin, as used herein, refers to the extracellular amino acids of one chain that interact with the second chain that comprises the integrin when the integrin is in an inactive state, but not when the integrin is in an active state. Stated another way, the extracellular clasp of an α chain comprises the extracellular amino acids of the α chain that interact with the β chain when the integrin is in an inactive state, but not when the integrin is in an active state. Alternatively, the extracellular clasp of a β chain comprises the extracellular amino acids of the β chain that interact with the α chain when the integrin is in an inactive state, but not when the integrin is in an active state. The amino acid residues of either chain of an integrin clasp may or may not be contiguous in the peptide chain constituting the integrin subunit. Further, in some embodiments, the alteration of the chemical properties of one or more amino acids constituting said clasp, by mutational analysis, may result in an integrin that is more readily activated than the integrin comprised of its native amino acid sequence.

In some embodiments, the α chain extracellular clasp region is comprised of an amino acid sequence listed in Table B. In one embodiment, the α chain extracellular clasp region comprises amino acids 331 to amino acid 338 of the αv integrin, i.e. MDRGSDGK (SEQ ID NO: 1). In another embodiment, the α chain extracellular clasp region comprises amino acids 345 to amino acid 352 of the αIIb integrin, i.e. MESRADRK (SEQ ID NO: 2).

TABLE B

| | | |
|---|---|---|
| αV | MDRGSDGK | SEQ ID NO:1 |
| αIIb | MESRADRK | SEQ ID NO:2 |
| α5 | MDRTPDGR | SEQ ID NO:3 |
| α8 | MEREFESN | SEQ ID NO:4 |

The β chain extracellular clasp region is generally comprised of an amino acid sequence listed in Table C. In one embodiment, the β chain extracellular clasp region comprises the amino acid sequence CTTRTDTC (SEQ ID NO: 5). In another embodiment, the β chain extracellular region comprises the amino acid sequence CERTTEGC (SEQ ID NO: 6).

TABLE C

| | | |
|---|---|---|
| β3 | CTTRTDTC | SEQ ID NO:5 |
| β2 | CERTTEGC | SEQ ID NO:6 |
| β6 | CTTSTDSC | SEQ ID NO:7 |
| β1 | CSLDTSTC | SEQ ID NO:8 |
| β5 | CSTDISTC | SEQ ID NO:9 |
| β7 | CSGDMDSC | SEQ ID NO:10 |
| β4 | CPLSNATC | SEQ ID NO:11 |
| β8 | CPSAAA(Q/H)C | SEQ ID NO:12 |

### I. Compounds for Modulating Integrin Activity

One aspect of the present invention encompasses a compound that modulates integrin activity. The term “modulates”, in this context, refers to either increasing or decreasing the activity of an integrin. Generally speaking, a compound of the invention modulates the activity of an integrin by altering the interaction between the α and β chain extracellular clasp regions of the integrin. The term “altering”, as used herein, refers to either stabilizing or disrupting the interaction between the α and β chain extracellular clasp regions. Typically, a compound that disrupts the extracellular clasp of an integrin will increase the activity of the integrin, i.e. activate the integrin. Conversely, a compound that stabilizes the extracellular clasp of an integrin will decrease the activity of an integrin.

A compound of the invention may be a peptide, an antibody, a small molecule, or any other compound that alters the interaction between the α and β chain extracellular clasp regions of an integrin.

### (a) Peptides

In one embodiment, the invention encompasses a peptide compound that modulates the activity of an integrin. Stated another way, the invention encompasses a peptide that alters the interaction between the α and β chain extracellular clasp regions of the integrin. For instance, a peptide may disrupt or stabilize the extracellular clasp region of an integrin. In one embodiment, a peptide of the invention will alter the interaction between the αv and β3 clasp regions. In another embodiment, a peptide of the invention will alter the interaction between the αIIb and the β3 clasp regions.

Generally speaking, the peptide may comprise the amino acid sequence of either the α chain clasp region or the β chain clasp region, or a portion thereof. In some embodiments, the peptide will comprise the amino acid sequence of the αv clasp region, or a portion thereof. In other embodiments, the peptide will comprise the amino acid sequence of the αIIb clasp region, or a portion thereof. In certain embodiments, the peptide will comprise the amino acid sequence of the β3 clasp region, or a portion thereof. In additional embodiments, the peptide will comprise the amino acid sequence of the β2 clasp region, or a portion thereof. In a further embodiment, the peptide may comprise an amino acid sequence listed in Table B or C, or a fragment thereof. In an exemplary embodiment, the peptide may comprise the amino acid sequence TTRTDTC (SEQ ID NO: 13). In another exemplary embodiment, the peptide may comprise the amino acid sequence YMESRADRK (SEQ ID NO: 14).

Usually, a peptide of the invention is at least 2, 3, 4, 5, 6, 7, 8, 9, 10, 11, 12, 13, 14, or 15 amino acids in length. In some embodiments, a peptide of the invention is more than 15 amino acids in length.

In each of the above embodiments, a peptide of the invention alters the interaction between the α and β chain extracellular clasp regions of an integrin. Assays for determining whether a peptide alters the interaction between the α and β chain clasp regions are known in the art and are detailed in the examples.

Methods of producing peptides of the invention are known in the art. For instance, the peptides may be synthesized, purified, and verified by mass spectrometry as described in McDonald, 2004.

Methods of preparing compositions of peptides suitable for administration to a subject are known in the art. For instance, see U.S. Pat. No. 6,086,918.

### (b) Antibodies

In another embodiment, the invention encompasses an antibody compound that modulates the activity of an integrin. Stated another way, the invention encompasses an antibody that alters the interaction between the α and β chain extracellular clasp regions of the integrin. For instance, an antibody may disrupt or stabilize the extracellular clasp region of an integrin. In one embodiment, an antibody of the invention will alter the interaction between the αv and β3 clasp regions. In another embodiment, an antibody of the invention will alter the interaction between the αIIb and the β3 clasp regions.

Usually, the antibody will recognize an epitope comprising the amino acid sequence of either the α chain clasp region or the β chain clasp region, or a fragment thereof. In some embodiments, the antibody may recognize an epitope comprising the amino acid sequence of the αv clasp region, or a fragment thereof. In other embodiments, the antibody may recognize an epitope comprising the amino acid sequence of the αIIb clasp region, or a fragment thereof. In certain embodiments, the antibody may recognize an epitope comprising the amino acid sequence of the β3 clasp region, or a fragment thereof. In additional embodiments, the antibody may recognize an epitope comprising the amino acid sequence of the β2 clasp region, or a fragment thereof. In a further embodiment, the antibody may recognize an epitope comprising an amino acid sequence listed in Table B or C, or a portion thereof. In an exemplary embodiment, the antibody may recognize an epitope comprising the amino acid sequence CTTRTDTC (SEQ ID NO: 15), or a portion thereof. In another exemplary embodiment, the antibody may recognize an epitope comprising the amino acid sequence MESRADRK (SEQ ID NO: 2), or a portion thereof.

In each of the above embodiments, an antibody of the invention alters the interaction between the α and β chain extracellular clasp regions of an integrin. Assays for determining whether an antibody alters the interaction between the α and β chain clasp regions are known in the art. For instance, the cell migration assay detailed in the examples may be used.

Methods of producing antibodies are known in the art. The term “antibody,” as used herein, refers to monoclonal antibodies, polyclonal antibodies, chimeric antibodies, humanized antibodies, fully human antibodies, or antibody fragments that comprise the epitope binding domain of the intact antibody, such as Fab fragments or single chain engineered and optimized antibody “mimetics”.

Methods of preparing compositions comprising antibodies suitable for administration to a subject are known in the art.

### (c) Small Molecules

In yet another embodiment, the invention may encompass a small molecule compound that modulates the activity of an integrin. Stated another way, the invention may encompass a small molecule that alters the interaction between the α and β chain extracellular clasp regions of the integrin. For instance, a small molecule may disrupt or stabilize the extracellular clasp region of an integrin. In one embodiment, a small molecule of the invention will alter the interaction between the αv and β3 clasp regions. In another embodiment, a small molecule of the invention will alter the interaction between the αIIb and the β3 clasp regions.

Assays for determining whether a small molecule alters the interaction between the α and β chain clasp regions are known in the art. For instance, the cell migration assay detailed in the examples may be used. Alternatively the binding of presently identified LIBS antibodies may be used, for example, in high throughput screening assays, to identify compounds that cause the subject integrin to become activated (alter its conformation in a manner consistent with known parameters of activation).

Methods of producing and screening small molecules are known in the art. Small molecules of the invention may exist in tautomeric, geometric or stereoisomeric forms. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures of R and S forms for each stereocenter present.

In a further embodiment, the small molecules of the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine- (N-methylglucamine) and procaine. All of these salts may be prepared by conventional means from the corresponding compound by reacting, for example, the appropriate acid or base with the any of the compounds of the invention.

### (d) Pharmaceutical Compositions

The compounds of the present invention may be formulated into pharmaceutical compositions and administered by a number of different means that will deliver a therapeutically effective dose. Such compositions may be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Solid dosage forms for oral administration may include capsules, tablets, pills, powders, and granules. In such solid dosage forms, the compound is ordinarily combined with one or more adjuvants appropriate to the indicated route of administration. If administered per os, the compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of the compound of the invention that may be combined with the carrier materials to produce a single dosage of the composition will vary depending upon the patient and the particular mode of administration. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### II. Modulating the Activity of an Integrin

Another aspect of the present invention encompasses methods for modulating the activity of an integrin. Typically, the method comprises altering the interaction between the α and β chain extracellular clasp regions of an integrin. In one embodiment, the activity of the integrin is increased. In another embodiment, the activity of the integrin is decreased. In yet another embodiment, the interaction between the α and β chain is stabilized. In still another embodiment, the interaction between the α and β chain is destabilized.

In some embodiments, the interaction between the a and D chain clasp regions may be altered with a compound of the invention described in section I above. For instance, the interaction may be altered by contacting the integrin with a peptide, an antibody, a small molecule, or any other compound that alters the interaction between the α and β chain extracellular clasp regions of an integrin.

In other embodiments, the interaction between the α and β chain clasp regions may be altered by altering a clasp region of the integrin. For instance, the α chain may be altered such that the altered α chain stabilizes or destabilizes the interaction between the α and β chain extracellular clasp. Alternatively, the β chain may be altered such that the altered β chain stabilizes or destabilizes the interaction between the α and β chain extracellular clasp. In one embodiment, for instance, the amino acids comprising the β3 chain clasp may be altered to comprise TTDTRT (SEQ ID NO: 15) (as opposed to the wild-type amino acid sequence TTRTDT (SEQ ID NO: 16)). In another embodiment, the αv chain clasp may be altered to introduce either or both of the mutations R303D and D306R.

The methods of the invention encompass modulating the activity of the αvβ3 integrin. In some embodiments, the activity of the αvβ3 integrin is increased. In other embodiments, the activity of the αvβ3 integrin is decreased. The activity of the αvβ3 integrin may be modulated by altering the interaction between the αv and β3 extracellular clasp regions. The interaction between the αv and β3 clasp regions may be altered by contacting the integrin with a compound described in section I above.

The methods of the invention encompass modulating the activity of the αIIbβ3 integrin. In some embodiments, the activity of the αIIbβ3 integrin is increased. In other embodiments, the activity of the αIIbβ3 integrin is decreased. The activity of the αIIbβ3 integrin may be modulated by altering the interaction between the αIIb and β3 extracellular clasp regions. The interaction between the αIIb and β3 clasp regions may be altered by contacting the integrin with a compound described in section I above.

### (a) Modulating Inflammation

In certain embodiments, the invention provides a method of modulating inflammation. Typically, the method comprises modulating the activity of an integrin by altering the interaction between the α and β chain extracellular clasp regions, as described above. Generally speaking, integrins play a central role in inflammation. Consequently, disrupting the interaction between the α and β chain clasp regions, may increase inflammation. Alternatively, stabilizing the interaction between the α and β chain clasp regions may decrease inflammation. Decreasing inflammation, may, in turn, reduce swelling, pain, or inflammation associated conditions. Additionally, increasing inflammation may, in turn, assist an immune response, as in individuals exhibiting an immunocompromised state.

In some embodiments, a peptide as described in section I(a) above may be used in a method for modulating inflammation. Methods for monitoring inflammation are known in the art and include measuring cytokine production and/or cell proliferation.

### (b) Modulating Angiogenesis

In several embodiments, the invention provides a method of modulating angiogenesis. Typically, the method comprises modulating the activity of an integrin by altering the interaction between the α and β chain extracellular clasp regions, as described above. Generally speaking, integrins play a central role in angiogenesis. For instance, see Brian P. Eliceiri and David A. Cheresh, J Clin Invest (1999) 103(9):1227-1230. Consequently, disrupting the interaction between the α and β chain clasp regions may increase angiogenesis. Alternatively, stabilizing the interaction between the α and β chain clasp regions may decrease angiogenesis. Decreasing angiogenesis, may, in turn, decrease tumor growth. Additionally, increasing angiogenesis may, in turn, increase the survival of new tissue growth.

In some embodiments, a peptide as described in section I(a) above may be used in a method for modulating angiogenesis. In one embodiment, a peptide comprising the αv or β3 extracellular clasp region, or a portion thereof, may be used in a method for modulating angiogenesis.

Methods for monitoring angiogenesis are known in the art and include the chick chorioallantoic membrane assay, corneal pocket assay, Matrigel implant assay, tumor vascularity, growth assays and others known to the art. For more details, see Brian P. Eliceiri and David A. Cheresh, J Clin Invest (1999) 103(9):1227-1230.

### (c) Modulating Cell Migration

In various embodiments, the invention provides a method of modulating cell migration. Typically, the method comprises modulating the activity of an integrin by altering the interaction between the α and β chain extracellular clasp regions, as described above. Generally speaking, integrin activation is necessary for cell migration. Therefore, disrupting the interaction between the α and β chain clasp regions may increase cell migration. Alternatively, stabilizing the interaction between the α and β chain clasp regions may decrease cell migration. Non-limiting examples of cell migration include tumor cell migration and inflammatory cell migration.

In some embodiments, a peptide as described in section I(a) above may be used in a method for modulating cell migration. Methods for monitoring cell migration are known in the art, for instance, see the Examples below.

### (d) Modulating Platelet Aggregation

The invention also provides a method for modulating platelet aggregation. Typically, the method comprises modulating the activity of an integrin by altering the interaction between the α and β chain extracellular clasp regions, as described above. In particular embodiments, the interaction between the αIIb and the β3 chain clasp regions may be altered. The activation of the αIIbβ3 integrin is a necessary step in platelet aggregation. Therefore, disrupting the interaction between the αIIb and the β3 extracellular clasp regions may increase platelet aggregation, and therefore, in turn, increase thrombis formation. Alternatively, stabilizing the interaction between the αIIb and the β3 extracellular clasp regions may decrease platelet aggregation, and therefore, in turn, decrease thrombus formation.

In some embodiments, a peptide as described in section I(a) above may be used in a method for modulating platelet aggregation. In one embodiment, a peptide comprising the αIIb extracellular clasp region may be used in a method for modulating platelet aggregation. For instance, a peptide comprising YMESRADRK (SEQ ID NO: 14) or a portion thereof may be used in a method for modulating platelet aggregation.

Methods for monitoring platelet aggregation are known in the art, and kits are available commercially, such as the SPAT™ kit from Analytical Control Systems, Inc. In addition methods based on ex vivo aggregometry are routinely used to assess platelet aggregation.

### (e) Modulating Osteoclast Activity

In several embodiments, the invention provides a method of modulating osteoclast activity. Typically, the method comprises modulating the activity of an integrin by altering the interaction between the α and β chain extracellular clasp regions, as described above. Generally speaking, integrins play a central role in osteoclast activity. For instance, see Nakamura I, et al., J Bone Miner Metab. 2007; 25(6):337-44. Consequently, disrupting the interaction between the α and β chain clasp regions may increase bone resorption. Alternatively, stabilizing the interaction between the α and β chain clasp regions may decrease bone resorption. Decreasing bone resorption, may, in turn, aid in osteoschlerosis. Additionally, increasing bone resorption may, in turn, aid in osteopetrosis.

In some embodiments, a peptide as described in section I(a) above may be used in a method for modulating osteoclast activity. In one embodiment, a peptide comprising the αv or β3 extracellular clasp region, or a portion thereof, may be used in a method for modulating osteoclast activity.

Methods for monitoring osteoclast activity are known in the art. For more details, see Nakamura I, et al., J Bone Miner Metab. 2007; 25(6):337-44.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Material and Methods

Reagents, cell lines and peptides—Human K562 erythroleukemic cells (ATCC: CCL-243), stably expressing αvβ3 integrin were maintained in RPMI 1640 media supplemented with 10% fetal bovine serum (FBS) (29). HEK 293 cells (ATCC: CRL1573) were maintained in DMEM with 10% FBS. C32 human melanoma cells (ATCC: CRL-1585) were maintained in MEM media with 10% fetal bovine serum. Ligand induced binding site (LIBS) antibodies, LIBS1 and LIBS6, were generously provided by Dr. Mark Ginsberg (Scripps Research Institute) (7) and the LIBS antibody D3 was a gift from Dr. Lisa Jennings (The University of Tennessee, Memphis, Tenn.) (9). FITC-anti-mouse IgG (Sigma-Aldrich, St. Louis Mo.) was employed as the secondary antibody for flow cytometry experiments. The following peptides were synthesized, purified and verified by mass spectrometry as previously described (30): β3 integrin, residues 561-567: P2483 (TTRTDTC; SEQ ID NO: 13), αIIb integrin, residues 313-321: P2484 (YMESRADRK; SEQ ID NO: 14), a control peptide (KMDASAAVS; SEQ ID NO: 17), 4N1K (KRFYVVMWKK; SEQ ID NO: 18), and GRGDSP (SEQ ID NO: 19). All other reagents were purchased from Sigma-Aldrich unless otherwise stated.

Cell Spreading Assay—Spreading of C32 cells on Vn was performed as described (31) in 24-well tissue culture plates coated with 0.5 μg/ml Vn and blocked with 1% BSA/PBS for 2 hrs at room temperature. C32 cells were plated in HBSS with 2 mM CaCl₂ and 2 mM MgCl₂ in the presence of 2 mM MnSO₄, and indicated amounts of GRGDSP (SEQ ID NO: 19), P2483, P2484, or 4N1K. Cells were allowed to spread for 30 minutes at 37° C., after which the cells were fixed, stained, and photographed.

Preparation of Fresh Platelets—Collection of human blood was performed under an approved protocol of the Washington University School of Medicine Human Studies Committee. 30 mL of blood was drawn from a healthy donor into 3% sodium citrate, and centrifuged at 200×g for 10 min. at room temp. to yield platelet-rich plasma (PRP). The PRP was treated with 1 μg/ml prostaglandin E1 (PGE-1) and centrifuged at 500×g for 5 min. The platelet pellet was resuspended in Tyrode's solution (137 mM NaCl, 2.7 mM KCl, 2 mM CaCl₂, 1 mM MgCl₂, 3.3 mM NaH₂PO₄, 5.5 mM glucose, 20 mM HEPES, pH 7.4 with 1 mg/ml BSA and 1 μg/ml PGE-1. The platelet suspension was stored on ice for 1 to 48 hr or treated as described below.

Flow Cytometry—Binding of anti-β3 mAb AP3 and anti-LIBS mAbs LIBS1, LIBS6 and D3 was quantified using a Coulter EPICS flow cytometer. 10⁶ cells were washed and resuspended in 100 μl of FACS buffer (1% BSA, 2 mM MgCl₂, in PBS) and incubated on ice for 30 minutes. Cells were washed and resuspended in buffer (PBS+2 mM MgCl₂ or 1 mM each CaCl₂ and MgCl₂) along with combinations of 0-1 mM GRGDSP (SEQ ID NO: 19) with 500 μM MnCl₂, 100 μM P2483, 100 μM P2484, and 100 μM P2485 or control peptide. Cells were then incubated on ice for 30 minutes. Cells were washed and incubated in 100 μl of buffer (PBS+2 mM MgCl₂) containing donkey anti-mouse IgG-FITC at a 1:100 dilution for an additional 30 min. After washing, the cells were diluted to 0.5 ml and analyzed by flow cytometry. Data was analyzed using WinMDI software.

Preparation and expression of mutants of αv and β3-Full length cDNA clones of human αv and β3 integrin subunits were provided by Dr. Scott Blystone. Restriction fragments containing the mutation sites were subcloned into Bluescript BSKS+ for PCR mutagenesis using overlapping primers containing the mutant bases. After confirmation by DNA sequencing, the restriction fragment containing the desired mutation was reassembled in either pCDNA3 for αv (G418 selection), and either pREP10 or pBLY100 for β3 (hygromycin selection). Initial tests of the β3 mutants were performed by transfecting them into human ovarian carcinoma clone OV10 which expresses WT αv (largely as αvβ5) but no β3 (29). For cotransfections of both subunits, 293 HEK cells were used (32) (33). Expression was determined by flow cytometry with mAbs L230 (αv) and AP3 (β3). LIBS binding was determined as above and normalized to AP3 binding or the binding of the LIBS mAb in the presence of excess RGDS peptide and Mn++ to yield an activation index.

Molecular dynamics simulations—The reported crystal structure of αvβ3 (PDB ID: 1JV2) (4) was subjected to energy minimization and equilibration using GROMACS version 3.3 (34). Details of the method are provided in Example 6.

### Example 1

### Clasp Peptides Stimulate Integrin Dependent Cell Adhesion and Spreading

As an initial approach to testing the function of the putative clasp region of β3 integrins, synthetic peptides were used to mimic the α and β sides of the clasp. It was reasoned that αIIbβ3 is likely to be held in the off state more securely than αvβ3, and thus the αIIb clasp sequence was chosen for these experiments (Table 1). To evaluate the effects of the putative clasp peptides on αvβ3 activation in live cells, adhesion and spreading assays using C32 melanoma cells were performed. We previously showed that, at relatively low densities of Vn coated on plastic (ca. 0.5 μg/ml), initial adhesion of C32 cells occurs via αvβ5. Not until αvβ3 is activated, e.g. via Mn⁺⁺ or CD47 stimulation, does αvβ3-dependent spreading occur (35). The assay was performed in Ca⁺⁺/Mg⁺⁺ which supports integrin activity, and we used Mn⁺⁺ activation of αvβ3 as a positive control (6). The putative clasp peptides were incubated with cells added to the Vn-coated wells and after 30 min at 37° cell attachment and spreading were assessed (FIG. 1). Mn⁺⁺ resulted in increased adhesion to Vn as well as enhanced spreading and 0.5 mM GRGDSP (SEQ ID NO: 19) effectively reduced cell adhesion.

TABLE 1
Comparison of the clasp regions of α and β integrin subunits.
α Subunits

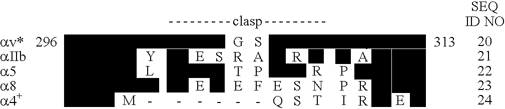

β Subunits

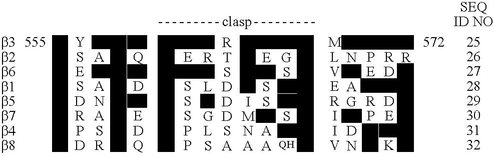

*αv pairs with β1, β3, β5, β6 and β8.
⁺α4 and all other integrin α subunits have deletions in the clasp region.

As another positive control, the C terminal TSP1 peptide, 4N1K, an agonist of CD47, was used, which, in this assay, signals αvβ3 activation via heterotrimeric Gi (36). C32 cell adhesion increased 400% in the presence of 50 μM 4N1K. Finally, the addition of 100 μM P2483 (β3 integrin peptide) or P2484 (αIIb integrin peptide) clasp peptides stimulated cell adhesion to an extent comparable to 4N1K (FIG. 1). Each of the treatments that increased cell adhesion to Vn also enhanced cell spreading (FIG. 1). Mn⁺⁺ and 4N1K increased the number of spread cells ca. 3-fold and 10-fold respectively, while addition of GRGDSP (SEQ ID NO: 19) nearly eliminated both adhesion and cell spreading. The β3 peptide increased cell spreading 9-fold, and the αIIb peptide increased cell spreading 78-fold.

Thus both clasp peptides were able to stimulate cell spreading, a signaling dependent function, much more effectively than Mn++. These results indicate that addition of either the αIIb or β3 clasp peptide is able to stimulate cell adhesion and spreading. While these are functions of activated αvβ3, the peptides might act indirectly via other non-integrin intermediaries to influence integrin dependent cell adhesion and spreading.

### Example 2

### Clasp Peptides Induce Conformation Changes in β3 Integrins Consistent with Activation

To more directly monitor changes in integrin conformation, a series of antibodies (mAbs) were used that react with ligand-bound or activated states of β3 integrins. These LIBS mAbs used here recognize epitopes at three different sites within the stalk region of the β3 subunit that are masked in the “off” state and only become accessible when the integrin is “opened up” in the ligand-binding, activated conformation (37). When K562 cells transfected to express αvβ3 integrin were incubated with the LIBS1 or LIBS6 mAbs alone (dotted histograms in FIG. 2), little antibody binding above isotype control mAb or secondary mAb alone was detected. D3 mAb appeared to induce some activation of the integrin since its binding to αvβ3 (FIG. 2, dotted line) was several fold above that of controls. GRGDSP (SEQ ID NO: 19) peptide augments LIBS binding by stabilizing active conformations of the β3 integrin. When 1 mM GRGDSP (SEQ ID NO: 19) peptide was added with LIBS1, LIBS6, or D3 antibodies (dashed histogram FIG. 2), an increase in fluorescence was seen compared to cells treated with LIBS antibody with no RGD peptide. This shift was further augmented by the addition of 0.5 mM Mn⁺⁺ ion. The effect of the clasp peptides on the binding of each LIBS mAb was determined in the presence of 1 mM RGD peptide, but in the absence of Mn⁺⁺.

Preliminary studies indicated that the peptide effects were maximal between 50 to 100 μM peptide under these conditions. As seen in FIG. 2, LIBS1 antibody binding was dramatically increased by both 100 μM P2483, the β3 clasp peptide (shaded histogram), and P2484, the αIIb clasp peptide (solid line), well beyond the increase in LIBS1 binding due to RGD peptide alone. This was seen in 8 of 8 experiments with LIBS1 (two of which were performed with OV10 cells transfected to express αvβ3) (38). In 6 of 8 experiments, rightward shifts with 2483 and 2484 peptides were greater than that promoted by RGD plus Mn⁺⁺. The control peptide did not enhance LIBS1 binding, nor did a number of other peptides based on sequences present in TSP1 or CD47 that were selected to have the same net charge as the clasp peptides.

In contrast to the results with LIBS1, the clasp peptides induced no additional binding of LIBS6 antibody beyond that achieved with RGD peptide (FIG. 2, representative of 3 experiments). However, the effect of Mn⁺⁺ itself was much less marked in the case of LIBS6 binding to αvβ3. In the case of the D3 LIBS mAb, RGD peptide resulted in an increase in D3 binding, and both clasp peptides further increased D3 binding (FIG. 2, representative of 3 experiments). Here the histogram shift induced by the clasp peptides was of about the same magnitude as that of Mn⁺⁺ (not shown). As expected from the fact that these three LIBS mAbs bind to different epitopes on β3, the effect of the clasp peptides was different for each mAb. Importantly, the effect of the α and β clasp peptides were comparable for each mAb, suggesting that they are acting via the same mechanism, i.e. competition for the endogenous clasp.

### Example 3

### Clasp Peptides Increase LIBS Antibody Binding to Platelet αIIbβ3

Platelets and megakaryocytes are the only cells to express αIIbβ3 integrin, and thus platelets offer a unique system in which to test the effects of the clasp peptides on β3 integrin activation. They also provide the opportunity to compare the activation response to the clasp peptides of αIIbβ3 vs αvβ3. Several G protein coupled receptors (e.g. those for ADP, thromboxane and thrombin) on platelets can rapidly activate αIIbβ3 via inside-out signaling (2). To eliminate this route of activation platelets were incubated with PGE1, which elevates cyclic AMP levels via Gs, and in some experiments also used apyrase to block activation by leaked ADP (39). In addition, platelets were kept on ice for as long as 24 to 48 hours to ensure metabolic inactivity. While short-term exposure to cold can activate platelets, long term exposure makes platelets refractory to activation (40). To be sure that platelets were metabolically inactive, they were challenged with 50 μM ADP or 10 μM thrombin receptor activating peptide (TRAP) in the presence of the 3 LIBS mAbs. Neither of these agonists was able to increase LIBS binding to the platelets used in these experiments, indicating that inside out signaling was effectively disabled.

The optimal concentration of RGD peptide needed to stabilize the activated state of αIIbβ3 was determined. The addition of 100 μM RGD was sufficient to increase LIBS1 binding, and this shift was further magnified with the addition of 0.5 mM Mn⁺⁺. As seen in FIG. 3, the β3 clasp peptide (P2483, shaded histogram) was unable to increase LIBS1 binding to αIIbβ3 and in some experiments, slightly reduced LIBS1 binding as compared to the effect of RGD alone. However, the αIIb peptide (P2484, solid line) increased LIBS1 binding in the presence of RGD to an extent comparable to the addition of Mn⁺⁺. In 6 experiments with LIBS1 (2 different platelet donors), the αIIb clasp peptide increased binding well above that with RGD alone, and in some cases to the extent seen with RGD plus Mn⁺⁺. In all 6 experiments, the β3 clasp peptide failed to increase LIBS1 binding. This suggests that the binding of the αIIb clasp to the β3 subunit is of substantially greater affinity than the binding of the αv clasp to β3. When flow cytometry was performed with LIBS6 antibody, results similar to those with αvβ3 were obtained in that neither clasp peptide increased binding of LIBS6 beyond that seen with RGD alone (FIG. 3). In the case of αIIbβ3 however, LIBS6 binding was not increased by addition of RGD at this concentration. This suggests that the epitope recognized by LIBS6 is more protected in αIIbβ3 than in αvβ3, likely due to the tighter “off state” of αIIbβ3.

Finally, the effect of the clasp peptides on the binding of D3 LIBS antibody to platelets (FIG. 3) was tested. Here, peptide P2483 (β3) marginally stimulated D3 binding, while P2484 (αIIb) produced large rightward shifts in the binding histogram, in this case even greater than those seen with Mn⁺⁺ plus RGD. Thus the αIIb clasp peptide is much more effective at activating αIIbβ3 than the β3 clasp peptide, while the two clasp peptides are more comparable in potency to activate αvβ3, again likely reflecting the difference in affinity of the two a clasps for the β3 subunit.

### Example 4

### Mutation of αvβ3 Clasp Residues

The data obtained with the peptides suggested that they compete for the endogenous integrin clasp and thus promote integrin activation. In order to confirm this notion, site-specific point mutations were sought in the αv and β3 clasps in the context of the full length integrin subunits. The crystal structure of αvβ3 (4) was used in an effort to identify specific amino acid residues in αv and β3 that might be important in stabilizing the clasp. In the crystal structure, the αv clasp peptide forms a loop that projects from the β-propeller domain. It contains the sequence RGSD (αv: 303-306) juxtaposed to the β3 clasp which contains the sequence RTD (β: 563-565), suggesting the possible formation of two R-D ion pairs or salt bridges, a situation analogous to the juxtamembrane intracellular α-β clasp (17) (41) (20). If the oppositely charged pairs do in fact form an extracellular clasp, then swapping the R and D in either αv or β3 should break the clasp since it would create an R-R and a D-D pairing. Springer's group mutated β3-R563 to insert a non-native Cys which formed a disulfide with the Cys inserted in place of αv-G307(6), indicating that this residue can make close contact with the αv clasp residues. Therefore, the β3-R563 and β3-D565 residues were initially focused on as candidates for mutation, and a full length β3 construct with the R and D swapped was created, forming the clasp sequence TTDTRT (SEQ ID NO: 15) (WT=TTRTDT; SEQ ID NO: 16). This swap mutation leaves unchanged the net charge and amino acid composition of this short segment of the peptide chain.

The β3R/D swap mutant was expressed in OV-10 cells (which lack β3 expression) where it paired with endogenous WT αv, resulting in cell surface expression of the integrin heterodimer with the β3 R/D swap mutation. To determine the activation status of the mutant integrin relative to WT αvβ3 expressed in the same cell line, the D3 LIBS mAb was employed and activation was expressed as the ratio of D3 bound at each RGD concentration to D3 binding in the presence of maximum RGD peptide and Mn⁺⁺. Normalization to binding of the conformationally insensitive mAb AP3 (33) gave the same results. Rather than using a single concentration of RGD peptide to stabilize the activated state of αvβ3, we determined the activation index over a concentration range of GRGDSP (SEQ ID NO: 19) to help identify differences in the inherent activation status of the integrin (42). As seen in FIG. 4A, the αvWTβ3R/D mutant became activated at a much lower concentration of RGD peptide than WT αvβ3, and achieved a higher activation index at 50 μM GRGDSP. In FIG. 4B, data from several experiments was pooled to assess the significance of the activation of the mutant vs WT integrin.

Data is shown herein for the low concentration range (0 to 10 μM) of RGD peptide where differences in activation index are most pronounced. A sensitive index of activation is the level of LIBS antibody binding in the presence of no RGD peptide. FIG. 4C shows that the binding (accessibility) of D3 to αvWTβ3R/D in the absence of RGD peptide was about 20 times the D3 binding to WT integrin. As a positive control for activation, a known β3-activating mutant was sought. The β3-T562N mutant was identified in a screen for activating mutations of αIIbβ3 (33). The β3 mutant was created and expressed it in OV10 cells where it paired with WT av. As seen in FIG. 4A, it was also activated at low concentrations of RGD peptide, similar to our β3R/D swap mutant (FIG. 4A). In 5 experiments, the αvWTβ3R/D mutant appeared slightly more activated at 0 to 10 μM RGD concentrations than the αvWTβ3T562N mutant.

Besides the β3-T562N mutant, two Glanzmann's mutations that activate αIIbβ3 have been mapped to β3-C560 (33, 43). However, none of these studies implicated interactions with the α subunit as the mechanism for increased integrin activation.

To test the effect of mutating the αv side of the clasp, the αvR/D swap mutant (R303D/D306R) was created and expressed with WT β3. This required using 293 HEK cells as expression hosts (33), since OV10 cells express high levels of WT αv subunit (normally paired with β5 in this cell type) (44). As with the β3 swap mutant, the αvR/Dβ3WT integrin was activated relative to the WT integrin as judged by the D3 activation index (FIG. 4A-C). Since differences in the expression host cell type can affect the activation status of β3 integrins (45), we also transfected 293 cells with plasmids encoding WT αv and the β3R/D mutant and found that αvWTβ3R/D exhibited identical activation behavior in 293 and OV10 cells. In fact, some of the experiments included in FIG. 4B were performed with the 293 transfectants and some with the OV10 cells. Thus both the αv and β3 R/D swap mutants result in activation, although the αvR/Dβ3WT integrin did not appear as readily activated as the αvWTβ3R/D. This is clearly seen in FIG. 4C where binding of D3 in the absence of RGD peptide is about half of that seen for αvWTβ3R/D.

If the main contribution to clasp stability were two R-D ion pairs, then one might expect that a “double swap” integrin, in which the R-Xn-D sequences had been swapped in both the αv and β3 chains, would have activation properties similar to WT αvβ3. To test this idea, the αvR/D and the β3R/D constructs were co-expressed in 293 cells, and the activation index of the double swap mutant was determined as a function of RGD peptide concentration as above. While not as activated as the αvWTβ3R/D integrin, the double swap integrin was activated to about the same extent as αvR/Dβ3WT (FIG. 4A-C). Thus introducing the R/D swap into the αv subunit partially reverses the activating effect of the β3 R/D swap, but it does not fully restore WT function. This result suggests that molecular interactions that stabilize the clasp are more complex than simple charge-charge interactions.

### Example 5

### Molecular Modeling of the Clasp Region of αvβ3

The αvβ3 crystal structure was solved at a resolution of 3.1 A (4) and at this resolution, one cannot determine the precise conformation of the residues in the clasp. In an attempt to arrive at a plausible structure for the clasp region, a molecular dynamics simulation approach was used to obtain an equilibrated structure for WT αvβ3. The equilibrated crystal structure is shown in FIG. 5 and FIG. 6. The clasp region of the structure is magnified in FIG. 5B to show the αv clasp that resides on a short loop projecting from the β-propeller domain of the αv chain (magenta) and the proximity of the β3 clasp (cyan). The αv chain clasp loop is deleted in many other integrin α subunits (Table 1). Since part of the minimization process is the assignment of hydrogen atoms, which are completely absent in the crystal data, the equilibrated structure contains plausible configurations for hydrogens covalently bound to amino acids in the clasp. The derived model reveals a complex interface between the αv and β3 chains in the clasp region (FIGS. 5C, 6, 7). In this equilibrated structure, the distance between the residues of the αv and β3 clasp residues is decreased overall compared to the crystal coordinates. The interface has many van der Waals contacts between side chains of residues as well as a complex network of hydrogen bonds involving serine and threonine residues of the clasp (FIGS. 7 and 8).

One of the three mutant integrins created, αv R/Dβ3 wt, was subjected to the same molecular dynamics protocol as WT αvβ3. Both the solvent-accessible surface area of the clasp interface and the distance between α and β subunit clasp residues were significantly (p<0.05) increased in the mutant integrin (FIG. 9, 10). These results are consistent with the increased activation state of the mutant relative to the WT heterodimer. Perhaps the most interesting result from the equilibrated WT structure is that none of the R or D residues in the α or β clasp segments pair in trans with the oppositely charged residues on the other chain. Instead, the β chain R563 and D565 are seen to pair with one another, closing a loop across a turn (FIG. 5C). In the α chain, D306 is seen paired in cis with K308, also forming an intrachain loop, while the side chain atoms of R303 make extensive van der Waals and apparent hydrogen-bonded contacts with both cis and trans Thr and Ser residues of the clasp. Thus in both chains of the clasp, the R and D residues may play important roles in organizing the three dimensional conformation of the clasp, but our simulations suggest that they do not form interchain salt bridges as initially hypothesized.

These data show that amino acid residues that are juxtaposed in the bent or genuflected state of the αvβ3 heterodimer seen in the crystal structure contribute to stabilization of the low affinity state of the integrin. The ability of the αIIb and β3 clasp peptides to activate αIIbβ3 indicates that this mechanism applies to αIIbβ3 as well. The enhanced activity of the integrins in the presence of either αIIb or β3 clasp peptides is evidenced in functional assays including cell adhesion and cell spreading. The most compelling evidence that the peptides are able to induce a conformational change in integrin structure is their effect on the binding of three different anti-LIBS antibodies, LIBS1, LIBS6 and D3, to both αvβ3 and αIIbβ3. Interestingly, the effect of the three peptides differs depending on which LIBS antibody is used and whether the integrin in question is αIIbβ3 or αvβ3. These results are expected since the three LIBS mAbs used here bind to distinct epitopes in the β3 stalk region. Furthermore, one expects that αIIbβ3 will be held in check more rigorously than αvβ3, a result further supported by the data.

### Example 6

### Comparison of Crystal Structure for αvβ3 and its Structure after Energy Minimization

The comparison of the crystal structure of αvβ3 and its energy minimized structure is shown as FIG. 6A. The αvβ3 structure after minimization revealed a complex interface at the clasp region of αv and β3 chains (FIG. 6B), which was different from the crystal structure obtained at 3.1 A resolution. The major differences include van der Waals contacts, hydrogen bond formation and electrostatic interactions in the clasp region as described below.

The Ramachandran plots for the crystal structure vs the minimized structure were compared (FIG. 7). The minimized structure clearly shows a more complete clustering of Φ/Ψ angles into “allowed” and favored regions of the plot.

The contact map for the residues in the clasp regions of the α and β chains is shown in FIG. 8. The contact map was generated with an embedded tool in VMD software. The interactions of residues at the interface of the clasp region were changed after energy minimization such that there is increased contact among αv residues 305 SER, 306 ASP and β3 residues 563 ARG, 564 THR, 565 ASP, 566 THR.

In addition to the increased contact at the clasp region for the αvβ3 structure, hydrogen bonds formed during the dynamics simulation of αvβ3 integrin (FIG. 9). The residues in the clasp region involved in forming the hydrogen bonds included ARG 303, SER 305 in the α chain, and THR 562, ARG 563 and ASP 565 in the β chain. There were no hydrogen atoms present in the crystal structure because of its 3.1 A resolution.

In addition to van der Waals contact and hydrogen bond formation, the electrostatic potential for the clasp regions of the crystal structure of αvβ3 and its minimized state was calculated with APBS electrostatics software. Results showed that there is a complex electrostatic interaction in the clasp region of αvβ3 that is likely to play an important role in the clasp region (FIG. 10) To understand how mutations might break the extracellular αvβ3 clasp and thus activate β3 integrins, the structural effect of mutations in the clasp regions of αvβ3 was studied with molecular dynamics simulations. The three mutations include: αv-R/D swap (R303D/D306R), β3-R/D swap (R563D/D565R) and the double swap (αv-R/D swap (R303D/D306R) co-expressed with β3-R/D swap (R563D/D565R)) as described in the methods above.

The area of contact and solvent accessible surface area at the clasp region were calculated for the wild type and three mutants of αvβ3 (FIG. 11 and FIG. 12). Results showed that the αv-R/D swap and double swap (αv-R/D swap and β3-R/D swap) increased the solvent accessible surface area and decreased the degree of contact (distance for contact set at less than 0.6 nm) in the clasp region compared to wild type. This indicated the clasp region between the α chain and β chain was separated by the mutations αv-R/D swap and double swap (αv-R/D swap and β3-R/D swap). Although experimental studies observed that the mutation of β3-R/D swap at the clasp region also activated the αv-β3 integrin, the number of contacts and the solvent accessible surface area were not significantly changed by the β3-R/D swap mutation. This might be related to the interrupted structure of the β chain caused by the missing residues at the EGF2 and EGF3 regions.

In addition to the solvent accessible surface area and the number of contacts at the clasp region, the effect of mutations in the clasp region were identified on the number of hydrogen bonds (FIG. 12). Results showed that the number of hydrogen bonds formed was changed at the clasp region of αvβ3 integrin by the three mutations. Although the mutation of β3-R/D swap did not significantly change the solvent accessible surface area and number of contacts at the clasp region, it changed the hydrogen bond network in the clasp region. This might explain why the β3-R/D swap mutation activated the αvβ3 integrin as observed in the experimental results.

In summary, molecular dynamics results supported the experimental observations that mutations in the clasp region lead to separation of the αV and β3 chains in the clasp. A major factor promoting the separation appears to be changes in the hydrogen bonding pattern in the clasp. Thus the results of both experiments and simulations support the idea that a complex interface exists at the clasp region of αvβ3 which could not be resolved in the crystal structure and which plays an important role in restraining the integrin in a functionally off state. Mutations of the clasp region that introduce changes in the hydrogen bond network, molecular contacts and electrostatic interactions between the α and β subunits perturb the clasp and allow for a more facile activation of the integrin.

### REFERENCES

1. Springer, T. A., Wang, J. H. (2004) Adv. Prot. Chem. 68, 29-63
2. Shattil, S. J. G., M. H., Brugge, J. S. (1994) Curr Opin Cell Biol 6(5), 695-704
3. Xiao, T., Takagi, J., Coller, B. S., Wang, J-H., Springer, T. A. (2004) Nature 432, 59-67
4. Xiong, J.-P., Stehle, T., Diefenbach, B., Zhang, R., Dunker, R., Scott, D. L., Joachimiak, A., Goodman, S. L., Arnaout, M. A. (2001) Science 294, 339-345
5. Xiong, J. P., Stehle, T., Zhang, R., Joachimiak, A. Frech, M., Goodman, S. L., Arnaout, M. A. (2002) Science 295, 151-155
6. Takagi, J., Petre, B. M., Walz, T., Springer, T. A. (2002) Cell 110, 599-611
7. Frelinger, A. L., Cohen, L., Plow, E. F., Smith, M. A., robers, J., Lam, S. C. T., Ginsberg, M. H. (1990) J Biol Chem 265, 6346-6352
8. Du, X., Gu, M., Weisel, J. W., Nagaswami, C., Bennett, J. S., Bowditch, R., Ginsberg, M. H. (1993) J Biol Chem 268, 23087-23092
9. Kouns, W. C., Wall, C. D., White, M. M., Fox, C. F., Jennings, L. K. (1990) J Biol Chem 265, 20594-20601
10. Alvarez, A. A., Axelrod, J. R., Whitaker, R. S., Isner, P. D., Bentley, R. C., Dodge, R. K., and Rodriguez, G. C. (2001) Gynecol Oncol 82(2), 273-278
11. Carman, C. V., Springer, T. A. (2003) Curr Opin Cell Biol 15, 547-556
12. Acevedo, L., Yu, J., Erdjument-Bromage, H., Miao, R. Q., Kim, J. E., Fulton, D., Tempst, P., Strittmatter, S. M., and Sessa, W. C. (2004) Nat Med 10(4), 382-388
13. Brown, E. J., Frazier, W. A. (2001) Trends in Cell Biol 11, 130-135
14. Tarui, T., Akakura, N., Majumdar, M., Andronicos, N., Takagi, J., Mazar, A. P., Bdeir, K., Kuo, A., Yarovoi, S. V., Cines, D. B., and Takada, Y. (2006) Thromb Haemost 95(3), 524-534
15. Zent, R., Fenczik, C. A., Calderwood, D. A., Liu, S., Dellos, M., and Ginsberg, M. H. (2000) J Biol Chem 275(7), 5059-5064
16. Hemler, M. E. (2005) Nat Rev Mol Cell Biol 6(10), 801-811
17. O'Toole, T. E., Katagiri, Y., Faull, R. J., Peter, K., Tamura, R., Quaranta, V., Loftus, J. C., Shattil, S. J., and Ginsberg, M. H. (1994) J Cell Biol 124(6), 1047-1059
18. Hughes, P. E., Diaz-Gonzalez, F., Leong, L., Wu, C., McDonald, J. A., Shattil, S. J., Ginsberg, M. H. (1996) J Biol Chem 271, 6571-6574
19. Partridge, A. W., Liu, S., Km, S., Bowie, J. U., Ginsberg, M. H. (2005) J Biol Chem 280, 7294-7300
20. Lu, C., Takagi, J., Springer, T. A. (2001) J Biol Chem 276, 14642-14648
21. Kim, M., Carman, C. V., Springer, T. A. (2003) Science 301, 1720-1725
22. Mehta, R. J., Diefenback, B., Brown, A., Cullen, E., Jonczyk, A., Gussow, D., Luckenbach, G. A., Goodman, S. L. (1998) Biochem. J. 330, 861-869
23. Du, X., Plow, E. F., Frelinger, A. L., III, O'Toole, T. E., Loftus, J. C., Ginsberg, M. H. (1991) Cell 65, 409-416
24. Nermut, M. V., Green, N. M., Eason, P., Yamada, S. S., Yamada, K. M. (1988) EMBO J 7, 4093-4099
25. Carrell, N. A., Fitzgerald, L. A., Steiner, B., Erickson, H. P., Phillips, D. R. (1985) J. Biol. Chem. 260, 1743-1749
26. Weisel, J. W., Nagaswami, C., Vilaire, G., Bennett, J. S. (1992) J Biol Chem 267, 16637-16643
27. Adair, B. D., Xiona, J-P., Maddock, C., Goodman, S. L., Arnaout, M. A., Yeager, M. (2005)
28. Beglova, N., Blacklow, S. C., Takagi, J., Springer, T. A. (2002) Nat Struct Biol 9, 282-287
29. Lindberg, F. P., Gresham, H. D., Reinhold, R. I., Brown, E. J. (1996) J Cell Biol 134, 1313-1322
30. McDonald, J. F., Zheleznyak, A., Frazier, W. A. (2004) J Biol Chem 279,
31. McDonald, J. F., Dimitry, J. M., Frazier, W. A. (2003) Biochemistry 42,
32. Bodary, S. C., Napier, M. A., and McLean, J. W. (1989) J Biol Chem 264(32), 18859-18862
33. Kashiwagi, H., Tomiyama, Y., Tadokoro, S., Honda, S., Shiraga, M., Mizutani, H., Handa, M., Kurata, Y., Matsuzawa, Y., Shattil, S. J. (1999) Blood 93, 2559-2568
34. Lindahl E, H. B. a. v. d. S. D. (2001) Journal of Molecular Modeling 7, 306-317
35. Gao, A.-G., Lindberg, F. P., Dimitry, J. M., Brown, E. J., Frazier, W. A. (1996a) J Cell Biol 135, 533-544
36. Frazier, W. A., Gao, A-G., Dimitry, J., Chung, J., Lindberg, F. P., Brown, E. J., Linder, M. E. (1999) J Biol Chem 274, 8554-8560
37. Honda, S., Tomiyama, Y., Pelletier, A. J., Annis, D., Honda, Y., Orchekowski, R., Ruggeri, Z., Kunicki, T. J. (1995) J Biol Chem 270,
38. Lindberg, F. P., Gresham, H. D., Schwarz, E., Brown, E. J. (1993) J Cell Biol 123, 485-496
39. Chung, J., Gao, A.-G., Frazier, W. A. (1997) J Biol Chem 272, 14740-14746
40. Peerschke, E. I., and Zucker, M. B. (1981) Blood 57(4), 663-670
41. Vinogradova, O., Velyvis, A., Velyviene, A., Hu, B., Haas, T. A., Plow, E. F., Qin, J. (2002) Cell 110, 587-597
42. O'Toole, T. E., Loftus, J. C., Du, X. P., Glass, A. A., Ruggeri, Z. M., Shattil, S. J., Plow, E. F., and Ginsberg, M. H. (1990) Cell Regul 1(12), 883-893
43. Ruiz, C., Liu, C. Y., Sun, Q. H., Sigaud-Fiks, M., Fressinaud, E., Muller, J. Y., Nurden, P., Nurden, A. T., Newman, P. J., Valentin, N. (2001) Blood 98,
44. Lindberg, F. P., Gresham, H. D., Reinhold, M. I., and Brown, E. J. (1996) J Cell Biol 134(5), 1313-1322
45. Han, J., Lim, C. J., Watanabe, N., Soriani, A., Ratnikov, B., Calderwood, D. A., Puzon-McLaughlin, W., Lafuente, E. M., Boussiotis, V. A., Shattil, S. J., and Ginsberg, M. H. (2006) Curr Biol 16(18), 1796-1806
46. Ginsberg, M. H., Partridge, A. and Shattil, S. J. (2005) Curr Opin Cell Biol 17, 509-516
47. Ma, Y. Q., Qin, J., and Plow, E. F. (2007) J Thromb Haemost 5(7), 1345-1352
48. Li, R., Mitra, N., Gratkowski, H., Vilaire, G., Litvinov, S. V., Nagasami, C., Weisel, J. W., Lear, J. D., DeGrado, W. F., Bennett, J. S. (2003) Science 300, 795-798
49. Luo, B. H., Springer, T. A., Takagi, J. (2004) PLoS Biol 2, 776-786
50. Kamata, T., Ambo, H., Puzon-Mclaughlin, W., Tieu, K., Handa, M. Ikeda, Y., Takada, Y. (2004) Biochem. J. 378, 1079-1082
51. Yan, B., Smith, J. W. (2001) Biochem 40, 8861-8867
52. Lu, C., Ferzly, M., Takagi, J., Springer, T. A. (2001) J Immunol 166, 5629-5637
53. Zhang, Q., Springer, T. A. I. (2001) J Biol Chem 276, 6922-6929
54. Hynes, R. O. (1992) Cell 69, 11-25
55. Hemler, M. E. (2003) Annu Rev Cell Dev Biol 19, 397-422

What is claimed is:

1. A compound that modulates the activity of an integrin by altering the interaction between the α and β chain extracellular clasp regions.

2. The compound of claim 1, wherein the compound is selected from the group of compounds consisting of a peptide, an antibody or fragment or mimetic thereof, and a small molecule.

3. The compound of claim 1, wherein the activity of the integrin is increased.

4. The compound of claim 1, wherein the activity of the integrin is decreased.

5. The compound of claim 1, wherein the interaction between the clasp regions is stabilized.

6. The compound of claim 1, wherein the interaction between the clasp regions is disrupted.

7. The compound of claim 1, wherein the α chain is either αIIb or αv.

8. The compound of claim 1, wherein the β chain is either β3 or β2.

9. The compound of claim 1, wherein the α chain is αIIb and the β chain is β3.

10. The compound of claim 1, wherein the α chain is αv and the β chain is β3.

11. The compound of claim 2, wherein the peptide comprises the amino acid sequence TTRTDTC (SEQ ID NO: 13) or YMESRADRK (SEQ ID NO: 14).

12. The compound of claim 1, wherein the compound modulates a condition selected from the group consisting of thrombosis, inflammation, angiogenesis, tumor cell migration, and osteoclast activity to inhibit or promote bone resorption.

13. A method of modulating the activity of an integrin, the method comprising altering the interaction of the α and β chain extracellular clasp region of the integrin.

14. The method of claim 13, wherein the activity of the integrin is increased.

15. The method of claim 13, wherein the activity of the integrin is decreased.

16. The method of claim 13, wherein the interaction between the clasp regions is stabilized.

17. The method of claim 13, wherein the interaction between the clasp regions is disrupted.

18. The method of claim 13, wherein the interaction is modulated by contacting the integrin with a compound of claim 1.

19. The method of claim 13, wherein modulating the activity of an integrin modulates a condition selected from the group consisting of thrombosis, inflammation, angiogenesis, tumor cell migration, and osteoclast activity to inhibit or promote bone resorption.
